(19) United States
(12) Patent Application Publication
(10) Pub. No.: US 2022/0000647 A1
(43) Pub. Date: Jan. 6, 2022

(54) STENT DELIVERY APPARATUS

(71) Applicant: OLYMPUS CORPORATION, Tokyo (JP)

(72) Inventor: Yuko KIRINO, Hachioji-shi (JP)

(73) Assignee: OLYMPUS CORPORATION, Tokyo (JP)

(21) Appl. No.: 17/480,617

(22) Filed: Sep. 21, 2021

Related U.S. Application Data
(63) Continuation of application No. PCT/JP2019/012673, filed on Mar. 26, 2019.

(51) Int. Cl. A61F 2/966

(52) U.S. Cl. CPC A61F 2/966; A61M 25/09; A61F 2002/9665

(57) ABSTRACT

A stent delivery apparatus includes a guide catheter through which a guide wire is inserted, a pusher catheter including a first lumen through which the guide catheter is inserted and a first communication hole communicating with the first lumen, a stent including a second lumen through which the guide catheter is inserted and a second communication hole communicating with the second lumen and disposed on a distal side of the pusher catheter, and a thread that connects the stent and the pusher catheter to each other in a releasable manner, wherein the thread has a first end portion that through the first communication hole, an intermediate portion that through the second communication hole, and a second end portion that extends around a part of the guide catheter located between a proximal end of the stent and a distal end of the pusher catheter.

### BACKGROUND OF THE INVENTION

### Field of the Invention

The present invention relates to a stent delivery apparatus.

This application is a continuation application based on a PCT International Application No. PCT/JP2019/012673, filed on Mar. 26, 2019. The content of the PCT International Application is incorporated herein by reference.

### Description of Related Art

A stent placement in a bile duct is known as a treatment for bile duct stenosis. Usually, the stent is attached to a stent delivery apparatus and introduced to a target part.

A stent delivery apparatus disclosed in U.S. Pat. No. 6,264,624 includes a stent, a guide catheter, and a pusher catheter. Since the stent is temporarily connected to the pusher catheter by a string, when the pusher catheter is pulled back, the stent is also pulled back to follow the pusher catheter, so that a placement position can be adjusted. When the guide catheter is retracted, the connection by the string is released and the stent can be released.

### SUMMARY OF THE INVENTION

In the stent delivery apparatus of U.S. Pat. No. 6,264,624, the stent and the pusher catheter are temporarily connected to each other by the guide catheter passing through a loop formed by the string. Since the loop is located in the stent, the loop reduces a gap between the stent and the guide catheter. As a result, it may interfere with movement of the guide catheter in the stent, which may cause an increase of the force required for releasing the stent.

Based on the above circumstance, an object of the present invention is to provide a stent delivery apparatus in which a force required for releasing a stent is less likely to increase while maintaining a structure capable of pulling back the stent.

### Solution to Problem

According to a first aspect of the present invention, a stent delivery apparatus includes: a guide catheter through which a guide wire is insertable; a pusher catheter having a distal end portion, a proximal end portion, and a lumen extending between the distal end portion and the proximal end portion, in which the guide catheter is inserted through the lumen; a stent disposed on a distal side with respect to a distal end of the pusher catheter by insertion of the guide catheter; and a string-shaped connection member having a loop shape and connecting the stent and the pusher catheter to each other in a releasable manner

The pusher catheter has a first communication hole communicating with the lumen on an outer peripheral surface of the distal end portion.

The stent has a distal end opening, a proximal end opening, a stent lumen that makes communication between the distal end opening and the proximal end opening, and a second communication hole provided on an outer peripheral surface on a distal end side with respect to the proximal end opening and communicating with the stent lumen. The stent is disposed such that the proximal end opening is located on the pusher catheter side.

In a state where the stent and the pusher catheter are connected to each other, the connection member has a first end portion that is supported by the pusher catheter through the first communication hole, an intermediate portion that is continued to the first end portion and extends to a space between the stent and the pusher catheter through the second communication hole, and a second end portion that is continued to the intermediate portion and extends around a part of the guide catheter located between a proximal end of the stent and the distal end portion of the pusher catheter.

According to a second aspect of the present invention, a stent delivery apparatus includes: a guide catheter through which a guide wire is insertable; a pusher catheter having a distal end portion, a proximal end portion, and a lumen extending between the distal end portion and the proximal end portion, in which the guide catheter is inserted through the lumen; a stent disposed on a distal side with respect to a distal end of the pusher catheter by insertion of the guide catheter; and a string-shaped connection member having a loop shape and connecting the stent and the pusher catheter to each other in a releasable manner.

One of the stent and the pusher catheter has a communication hole. The stent has a distal end opening, a proximal end opening, and a stent lumen that makes communication between the distal end opening and the proximal end opening. The stent is disposed such that the proximal end opening is located on the pusher catheter side.

In a state where the stent and the pusher catheter are connected to each other, the connection member has a first end portion that is supported by the other of the stent and the pusher catheter, an intermediate portion that is continued to the first end portion and extends to at least a space between the stent and the pusher catheter through the communication hole, and a second end portion that is continued to the intermediate portion and extends around a part of the guide catheter located on a hand side with respect to a proximal end of the stent.

### BRIEF DESCRIPTION OF THE DRAWINGS

FIG. 1 is an overall view of a stent delivery apparatus according to a first embodiment of the present invention.

FIG. 2 is a side view of a stent according to the stent delivery apparatus.

FIG. 3 is a diagram showing an internal structure of the stent.

FIG. 4 is a schematic cross-sectional view of the stent delivery apparatus.

FIG. 5 is an enlarged schematic view showing a connection part between the stent and a delivery catheter.

FIG. 6 is a cross-sectional view taken along the line I-I of FIG. 5.

FIG. 7 is a diagram showing a placed stent.

FIG. 8 is an enlarged schematic view showing a connection part between a stent and a delivery catheter in a stent delivery apparatus according to a second embodiment of the present invention.

FIG. 9 is a cross-sectional view taken along the line II-II of FIG. 8.

FIG. 10 is an enlarged schematic view showing a connection part between a stent and a delivery catheter in a stent delivery apparatus according to a third embodiment of the present invention.

FIG. 11 is a cross-sectional view taken along the line III-III of FIG. 10.

FIG. 12 is an enlarged schematic view showing a connection part between a stent and a delivery catheter in a modification example of the stent delivery apparatus.

FIG. 13 is a cross-sectional view taken along the line IV-IV of FIG. 12.

### DETAILED DESCRIPTION OF THE INVENTION

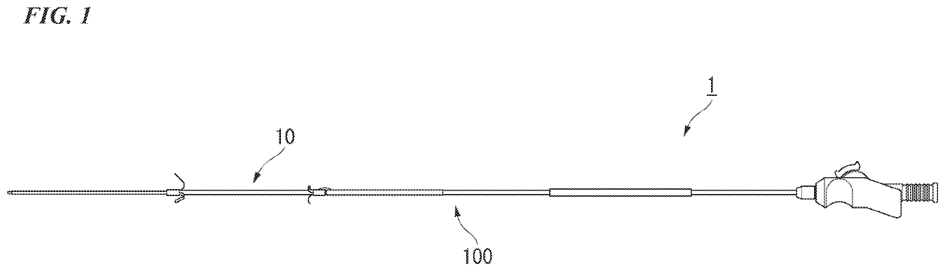

A first embodiment of the present invention will be described with reference to FIGS. 1 to 7.

FIG. 1 is an overall view of a stent delivery apparatus 1 of the present embodiment. The stent delivery apparatus includes a stent 10 and a delivery catheter 100.

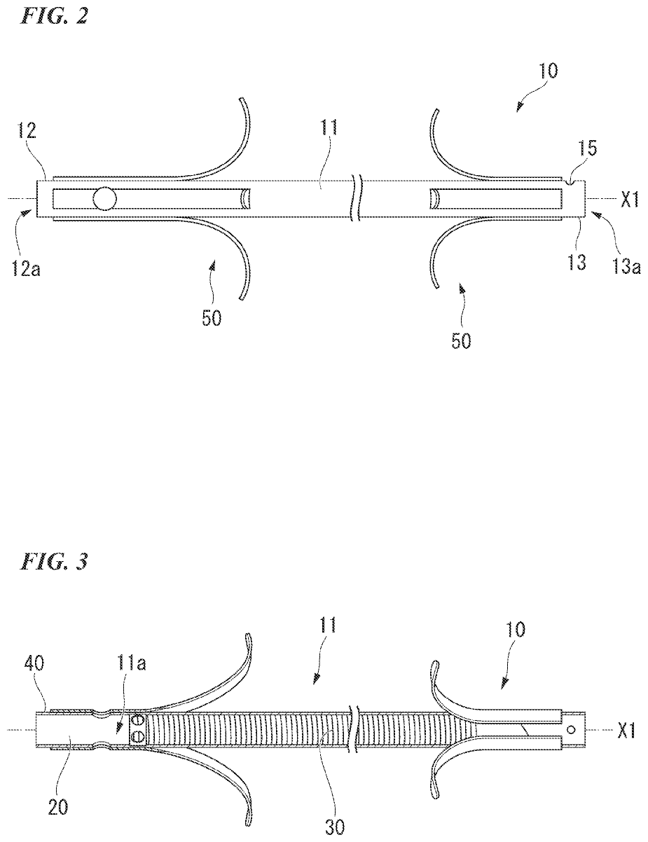

FIG. 2 is a side view of the stent 10. The stent 10 of the present embodiment is a stent to be placed in a bile duct, and includes a tubular main body 11 and flaps 50 attached to both end portions of the main body 11. The main body 11 has a distal end 12 having a distal end opening 12a and a proximal end 13 having a proximal end opening 13a, and extends along a longitudinal axis X1. A stent lumen 11a extends between the distal end opening 12a and the proximal end opening 13a along the longitudinal axis X1. The distal end 12 is an end portion that is disposed on a liver side when placed in the bile duct. The proximal end 13 is an end portion that is disposed on a duodenal papilla side when placed in the bile duct.

FIG. 3 is a diagram showing an internal structure of the stent 10. The main body 11 has a resin inner layer 20, a metal wire rod 30 wound around the inner layer 20, and a resin outer layer 40 covering the inner layer 20 and the wire rod 30. The wire rod 30 is embedded between an inner peripheral surface and an outer peripheral surface of the main body 11.

The inner layer 20 is a tube formed of a resin material such as polytetrafluoroethylene (PTFE) and perfluoroalkoxy alkane (PFA) having a smooth surface and biocompatibility.

The wire rod 30 is wound in a spiral shape on an outer peripheral surface of the inner layer 20, and is formed in a coil shape as a whole. A material of the wire rod 30 is a material having X-ray impermeableness, such as tungsten steel and stainless steel.

The outer layer 40 is formed of a resin material such as urethane or polyethylene having elasticity, flexibility, and biocompatibility. The outer layer 40 is also provided in a gap between the wire rods 30 adjacent to each other in a direction of the longitudinal axis X1.

A hole (second communication hole) 15 communicating with the stent lumen 11a is formed on an outer peripheral surface of one end portion of the stent 10. The hole 15 is used for a temporary connection (described below) between the stent 10 and the delivery catheter 100.

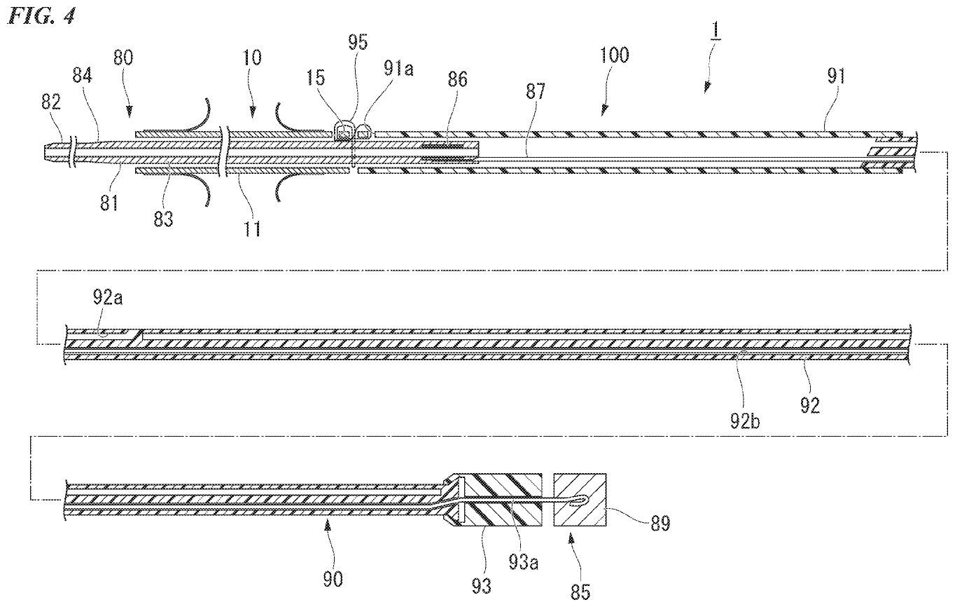

FIG. 4 is a schematic cross-sectional view showing a structure of the stent delivery apparatus 1. The delivery catheter 100 includes a guide catheter 80 and a pusher catheter 90.

The guide catheter 80 has a tube (guide tube) 81 through which a guide wire can be inserted, and a traction portion 85 for moving the tube 81.

The tube 81 is a tubular member made of resin and has a lumen through which a guide wire can be inserted. The tube 81 is flexible to such an extent that it is deformable when the tube 81 comes into contact with a living tissue during use of the stent delivery apparatus 1. The tube 81 is an elastic member having a restoring force, and becomes linear due to the restoring force in a state where no external force is applied. The tube 81 has a small diameter portion 82 located on a distal end side of the stent delivery apparatus 1 and a large diameter portion 83 located on a proximal end side of the stent delivery apparatus 1. A part where an outer peripheral surface of the small diameter portion 82 and the large diameter portion 83 are connected to each other is formed as a tapered intermediate portion 84, and the small diameter portion 82 and the large diameter portion 83 are connected to each other without difference in level. As a result, the outer diameter of the tube 81 gradually increases from the small diameter portion 82 toward the large diameter portion 83.

Outer diameters of the small diameter portion 82 and the large diameter portion 83 are smaller than an inner diameter of the stent 10. Therefore, the tube 81 can be inserted into the stent 10.

A material of the tube 81 is made of a fluororesin, a thermoplastic resin, or the like, and the following can be exemplified. The material of the tube 81 is not particularly limited as long as desired mechanical properties are satisfied.

General-purpose resins such as olefin resins such as polypropylene and polyethylene, copolymer resins thereof, polyester resins such as polyethylene terephthalate (PET) and polybutylene terephthalate (PBT), and polyvinyl alcohol (PVA).

Engineering resins such as polyamide resins, fluorine resins (for example, polytetrafluoroethylene (PTFE), polyvinylidene fluoride (PVDF), PFA, PEP, or ETFE), and polyetheretherketone (PEEK).

In addition, various elastomer resins (polystyrene, polyolefin, polyurethane, polyester, polyamide, polyvinyl chloride, and the like), silicone-containing resins, polyurethane-based resins, and the like.

The traction portion 85 includes a pipe 86, a wire 87, and an operation part 89. The pipe 86 is a metal tubular member having both ends open in an axial direction. The pipe 86 is attached to an inside of the tube 81 coaxially with the tube 81. The pipe 86 is disposed at a proximal end portion of the large diameter portion 83.

As a material of the pipe 86, a metal such as stainless steel and an engineering resin such as PEEK are exemplary examples, but other materials may be used as long as desired mechanical properties are satisfied.

A distal end portion of the wire 87 is joined to the pipe 86, and a proximal end portion thereof is connected to the operation part 89.

As a material of the wire 87, the same material as that of the pipe 86 is an exemplary example. Other materials may be used as long as desired mechanical properties are satisfied.

The pusher catheter 90 has a single lumen tube 91, a multi-lumen tube 92, and a grip portion 93.

The single lumen tube 91 is a tubular member having an inner diameter into which the large diameter portion 83 of the tube 81 can be inserted. The single lumen tube 91 has flexibility. A distal end surface of the single lumen tube 91 is a plane orthogonal to a center line of the single lumen tube 91. The distal end surface of the single lumen tube 91 can support the stent 10 by abutting on a proximal end of the stent 10. A size of a wall thickness of the single lumen tube 91 is equal to or greater than a difference between an inner radius and an outer radius of the main body 11 of the stent 10 (that is, a wall thickness of the stent 10). The single lumen tube 91 has a length for allowing the large diameter portion 83 of the tube 81 to be completely accommodated inside the single lumen tube 91.

The multi-lumen tube 92 is fixed to a proximal end portion of the single lumen tube 91. The multi-lumen tube 92 has a communication passage 92a for inserting a guide wire therethrough and a wire lumen 92b. The wire 87 of the guide catheter 80 is inserted through the wire lumen 92b.

The communication passage 92a is open to a distal end of the multi-lumen tube 92 and is open to a side surface of the multi-lumen tube 92 on the proximal end side with respect to the distal end of the multi-lumen tube 92.

The wire lumen 92b is open to the distal end and the proximal end of the multi-lumen tube 92.

The grip portion 93 is connected to a proximal end portion of the multi-lumen tube 92. The grip portion 93 has a substantially cylindrical shape having a diameter larger than that of the multi-lumen tube 92. Unevenness or the like for preventing slipping may be formed on an outer peripheral surface of the grip portion 93.

A through-hole 93a communicating with the wire lumen 92b is formed in the grip portion 93. The through-hole 93a is located on an extension line to the proximal end side of a center line of the multi-lumen tube 92. The through-hole 93a may not be on the extension line of the center line of the multi-lumen tube 92.

The wire 87 of the guide catheter 80 is inserted through the through-hole 93a. Thus, the wire 87 and the operation part 89 extend from the through-hole 93a.

The single lumen tube 91 and the multi-lumen tube 92 can be suitably used such that the kinds of materials to be blended are the same and only a blending ratio is different. In this case, when both are welded and joined, it is easy to adjust desired bending rigidity while maintaining a joining strength.

As a resin material of the single lumen tube 91 and the multi-lumen tube 92, the same resin as that of the tube 81 can be used. For example, when a relatively soft elastomer resin and a relatively hard thermoplastic resin are blended and a blending ratio of the thermoplastic resin in the multi-lumen tube 92 is made higher than that in the single lumen tube 91, the bending rigidity of the single lumen tube 91 is made to be smaller than the bending rigidity of the multi-lumen tube 92, and the insertability of the delivery catheter 100 can be enhanced.

The stent 10 is made to pass through the tube 81 projecting from the pusher catheter 90. The stent 10 is attached to the delivery catheter 100 with the end portion having the hole 15 located on the pusher catheter 90 side.

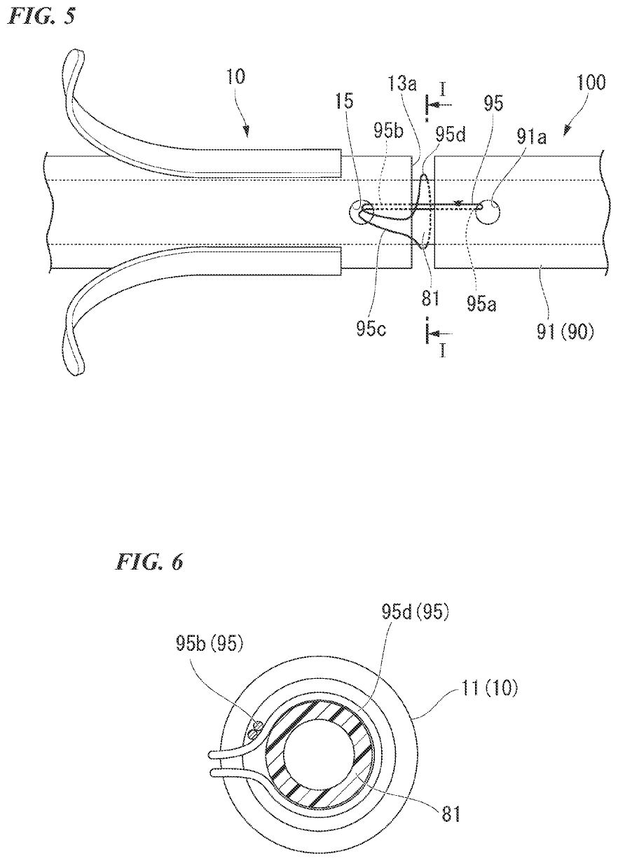

FIG. 5 is an enlarged schematic view showing a connection part between the stent 10 and the delivery catheter 100. FIG. 6 is a cross-sectional view taken along the line I-I of FIG. 5. A hole (first communication hole) 91a communicating with an internal space (lumen) is provided at a distal end portion of the single lumen tube 91. A thread (connection member) 95 is made to pass through the hole 91a. Since both end portions of the thread 95 are tied to form a loop shape in a state where the thread passes through the hole 91a, the thread is supported by the single lumen tube 91 and does not come off from the hole 91a.

The thread 95 having a loop shape enters the stent lumen 11a through the proximal end opening 13a of the main body 11 and exits the stent 10 from the hole 15 through a space between an inner surface of the stent 10 and an outer surface of the tube 81. The thread 95 exiting the hole 15 extends between the proximal end of the stent 10 and a distal end portion of the pusher catheter 90 and is disposed around the tube 81. That is, the tube 81 passes through the loop shape of the thread 95 between the stent 10 and the pusher catheter 90.

In the state shown in FIGS. 5 and 6, the thread 95 does not come off from the through-hole 15 unless the tube 81 comes off from the loop of the thread 95. As a result, the stent 10 and the delivery catheter 100 are temporarily connected to each other.

In a state where the stent 10 and the delivery catheter 100 are temporarily connected to each other, the loop shape formed by the thread 95 has, as shown in FIGS. 5 and 6, a first end portion 95a locked to the hole 91a and supported by the pusher catheter 90, a first intermediate portion 95b extending from the first end portion 95a to the hole 15 through the stent 10, a second intermediate portion 95c extending from the hole 15 to a space between the stent 10 and the pusher catheter 90 through the outside of the stent 10, and a second end portion 95d disposed around the tube 81 between the stent 10 and the pusher catheter 90. The second end portion 95d extends in a circumferential direction of the tube 81.

In a state where the stent 10 and the delivery catheter 100 are temporarily connected to each other, when the operation part 89 is pushed in to advance the guide catheter 80 to the maximum extent, the intermediate portion 84 of the tube 81 is exposed from the stent 10 held by the pusher catheter 90.

As a material of the thread 95, nylon is an exemplary example.

Dimensional examples of each part of the delivery catheter 100 are shown below, but the configuration of the present embodiment is not limited to this example.

Overall length of guide catheter 80: 2100 mm to 2300 mm

Length of tube 81: 350 mm to 450 mm

Length of wire 87: 1750 mm to 1850 mm

Overall length of pusher catheter 90: 1700 mm to 1800 mm

Overall length of single lumen tube 91: 480 mm to 520 mm

Overall length of multi-lumen tube 92: 1220 mm to 1280 mm

The operation of the stent delivery apparatus 1 configured as described above during use will be described with reference to an example of a case in which the stent 10 is placed in the bile duct.

An operator allows the guide wire to pass through a channel of a side-viewing endoscope and inserts the guide wire into the bile duct while observing it with the endoscope. Subsequently, the operator operates the guide wire under fluoroscopy to break through a stenotic part in the bile duct, and moves a distal end portion of the guide wire to the liver side from the stenotic part.

The operator inserts a proximal end portion of the guide wire projecting from a forceps opening of the endoscope into a distal end opening of the tube 81 of the stent delivery apparatus 1 to which the stent 10 is attached. The guide wire enters the lumen of the single lumen tube 91 through the proximal end opening of the tube 81. Further, the operator allows the proximal end portion of the guide wire to enter the wire lumen 92a and to project from a proximal end side opening of the wire lumen 92a.

The operator inserts the stent delivery apparatus 1 through which the guide wire is made to pass into the channel of the endoscope and allows a distal end portion of the stent delivery apparatus 1 to project from a distal end of the channel. The operator operates a raising base of the endoscope to direct the distal end of the stent delivery apparatus 1 toward the duodenal papilla and allows the stent delivery apparatus 1 to enter the bile duct along the guide wire. In a state where the stent 10 is positioned on the pusher catheter 90 by the thread 95, a distal end portion of the large diameter portion 83 of the tube 81 projects from the distal end of the stent 10. As shown in FIG. 4, since a difference between the outer diameter of the large diameter portion 83 and the inner diameter of the stent 10 is small, there is no large gap between the stent 10 and the tube 81, and an operation for breaking through the stenotic part is easy.

When a distal end portion of the stent 10 breaks through the stenotic part and the flap 50 on the distal end side moves to the liver side from the stenotic part, the operator advances and retracts the stent delivery apparatus 1 to determine a placement position of the stent 10. In the stent delivery apparatus 1, since the stent 10 and the delivery catheter 100 are temporarily connected to each other as described above, the stent 10 can be pulled back by retracting the stent delivery apparatus 1. Therefore, the position of the stent 10 can be easily adjusted.

After the placement position of the stent 10 is determined, the operator pulls the operation part 89 toward a hand side while holding the pusher catheter 90. Then, the wire 87 and the tube 81 retract, but the stent 10 does not retract because it is in contact with the pusher catheter 90. When the tube 81 retracts and comes off from the loop of the stent 10 and thread 95 (second end portion 95d), the connection between the stent 10 and the delivery catheter 100 is released and the stent 10 is placed at a desired position in the bile duct.

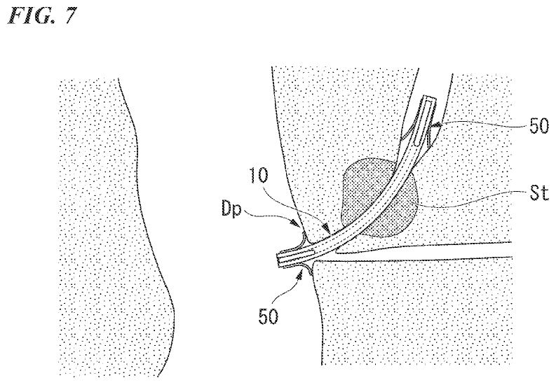

After the placement of the stent 10, as shown in FIG. 7, the flap 50 is placed on the liver side from a stenotic part St and near a duodenal papilla Dp outside the bile duct, so that the placement position is suitably held.

As described above, in the stent delivery apparatus 1 of the present embodiment, since the stent 10 and the delivery catheter 100 are temporarily connected to each other, the position of the stent 10 can be easily adjusted by the pull-back operation, and the stent can be placed at a desired position.

Further, in a state where the stent 10 and the delivery catheter 100 are temporarily connected to each other, the second end portion 95d extending in the circumferential direction of the tube 81 is located between the stent 10 and the pusher catheter 90. On the other hand, in the stent 10, only a portion of the thread 95 extends along the longitudinal axis in the gap between the stent 10 and the tube 81. Therefore, when the tube 81 is retracted with respect to the stent 10, the thread 95 is less likely to generate a large frictional resistance.

As described above, the stent delivery apparatus 1 of the present embodiment realizes a structure in which an operation force required for releasing the stent is less likely to increase even when a difference between an inner diameter dimension of the stent 10 and an outer diameter dimension of the tube 81 is small.

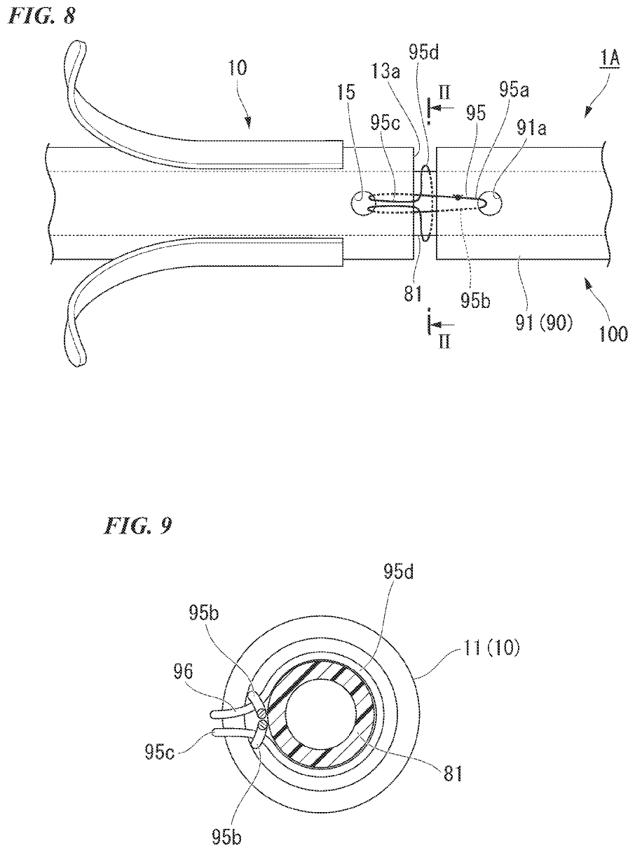

A second embodiment of the present invention will be described with reference to FIGS. 8 and 9. In the following description, the same reference numerals will be given to the same configurations as those described above, and a duplicate description thereof will be omitted.

FIG. 8 is an enlarged schematic view showing a connection part between the stent 10 and the delivery catheter 100 in a stent delivery apparatus 1A of the present embodiment. FIG. 9 is a cross-sectional view taken along the line II-II of FIG. 8.

As shown in FIGS. 8 and 9, the second intermediate portion 95c of the thread 95 extends between two threads of the first intermediate portion 95b. That is, the second end portion 95d passes through the loop shape of the thread 95, and a boundary portion 96 connecting the second intermediate portion 95c and the second end portion 95d is also located between the two threads of the first intermediate portion 95b.

In a side view from a direction orthogonal to the longitudinal axis of the delivery catheter 100, the boundary portion 96 is located between the proximal end of the stent 10 and the distal end portion of the pusher catheter 90 and crosses a space between the two threads of the first intermediate portion 95b.

The stent delivery apparatus 1A of the present embodiment also has the same effect as the stent delivery apparatus 1 of the first embodiment.

In addition, the boundary portion 96 is located between the two threads of the first intermediate portion 95b, and the second intermediate portion 95c is held by the first intermediate portion 95b. As a result, a tension generated by pulling the first intermediate portion 95b in the longitudinal direction during the pull-back operation of the stent 10 makes the second intermediate portion 95c hard to loosen when the stent 10 and the delivery catheter 100 are connected to each other.

Further, since the second intermediate portion 95c is hard to loosen, it is possible to prevent a situation in which a part of the second end portion enters the stent to increase the frictional resistance.

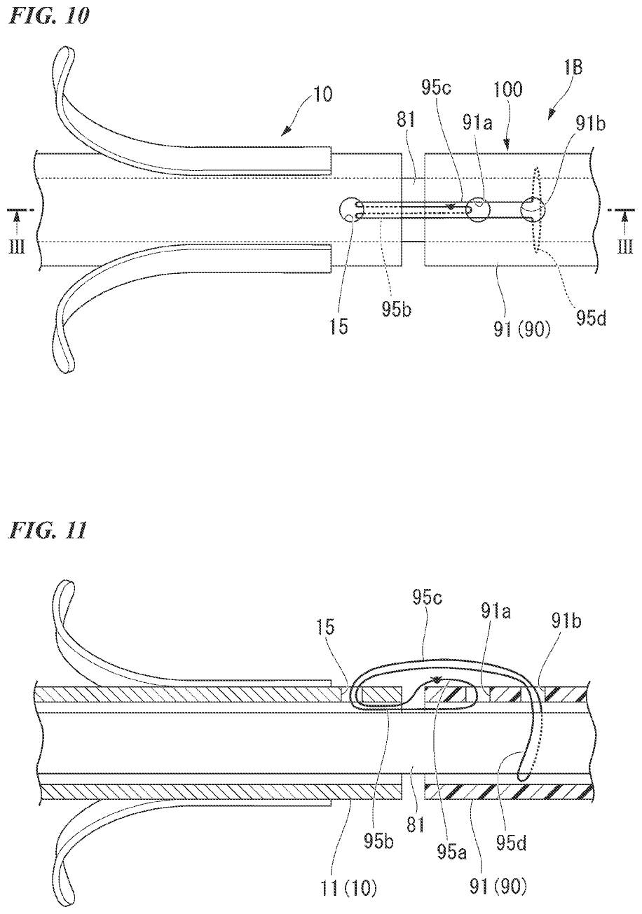

A third embodiment of the present invention will be described with reference to FIGS. 10 to 13.

FIG. 10 is an enlarged schematic view showing a connection part between the stent 10 and the delivery catheter 100 in a stent delivery apparatus 1B of the present embodiment. FIG. 11 is a cross-sectional view taken along the line III-III of FIG. 10, in which the thread 95 is slightly slackened in order to facilitate understanding of the disposition of the thread.

The single lumen tube 91 of the pusher catheter 90 has a hole 91b communicating with the lumen on the proximal end side with respect to the hole 91a. The second intermediate portion 95c of the thread 95 extends from the hole 15 of the stent 10 to the hole 91b. The thread 95 enters the inside of the single lumen tube 91 through the hole 91b and is disposed around the tube 81. That is, the second end portion 95d of the present embodiment is located in the pusher catheter 90.

In the stent delivery apparatus 1B of the present embodiment, an inner diameter of the pusher catheter 90 is larger than the inner diameter of the stent 10. Therefore, in a case where the second end portion 95d is disposed in the pusher catheter 90, a frictional resistance is less likely to occur than in a case where the second end portion 95d is disposed in the stent 10. Therefore, the stent delivery apparatus 1B of the present embodiment also has the same effect as the stent delivery apparatus 1 of the first embodiment.

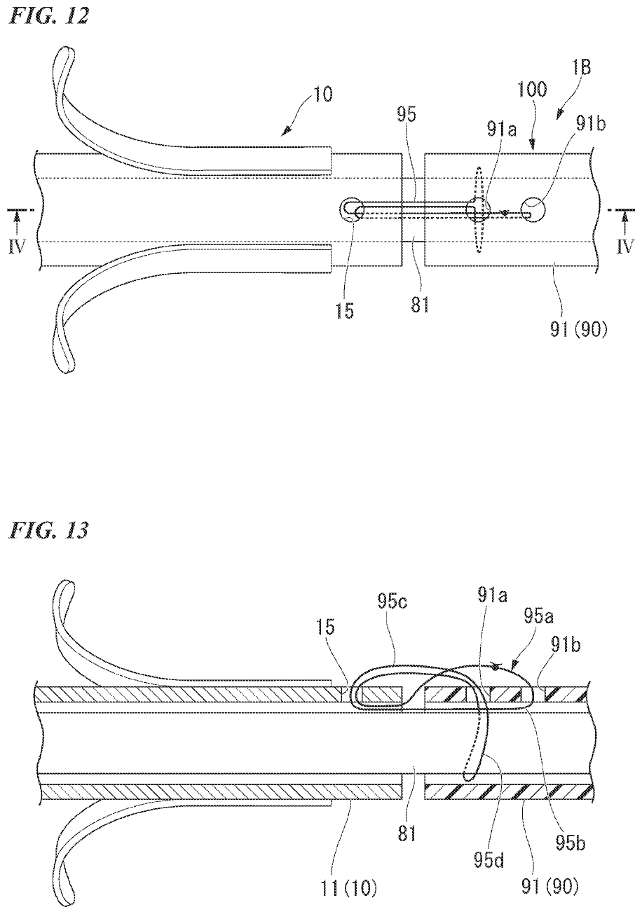

FIG. 12 is an enlarged schematic view showing a connection part between the stent 10 and the delivery catheter 100 in a modification example of the stent delivery apparatus 1B. FIG. 13 is a cross-sectional view taken along the line IV-IV of FIG. 12, in which the thread is slightly slackened as in FIG. 11.

In the modification example, the first end portion 95a of the thread 95 is located in the hole 91b. According to this, the first intermediate portion 95b extends from the hole 91b to the hole 15 of the stent 10. The second intermediate portion 95c extends from the hole 15 to the hole 91a. The thread 95 enters the inside of the single lumen tube 91 through the hole 91a and is disposed around the tube 81. Also in the modification example, since the second end portion 95d is located in the pusher catheter 90, the same effect as that of the stent delivery apparatus 1B is obtained.

As described above, the present invention is characterized in that the second end portion 95d extending in the circumferential direction of the tube 81 is not disposed in the stent 10.

Although each embodiment of the present invention has been described above, the technical scope of the present invention is not limited to the above-described embodiment, and it is possible to change the combination of components, to make various modifications to the components, and to delete the components without departing from the spirit of the present invention.

Hereinafter, some modifications are shown as examples, but the present invention is not limited thereto, and other modifications may be made.

In the first embodiment and the second embodiment, a positional relationship between the first communication hole and the second communication hole may be reversed. That is, the second communication hole may be provided in the pusher catheter and the first communication hole may be provided in the stent.

In this case, while the first end portion of the connection member is disposed in the stent and placed in the body, the connection member is located in the duodenum. Therefore, the stent can be extracted from the bile duct by pulling the connection member.

A method of forming the connection member into a loop shape is not limited to a method of connecting the end portions, and another method such as joining the end portions by welding or adhesion may be used.

The first end portion passed through the first communication hole may be fixed to an outer or inner surface of the pusher catheter or the stent. Further, the first end portion of the connection member does not necessarily have to pass through the first communication hole. For example, the first end portion may be fixed directly to the outer or inner surface of the pusher catheter or the stent. In this case, it is natural that the first communication hole may not be provided.

The flap of the stent is not limited to the aspect in which a separate member is attached to the main body as in the above example. For example, the flap may be formed by making a notch in the main body and bending a part of the main body. In this case, when the notch is made to reach the stent lumen, a communication hole is formed along with the formation of the flap. Therefore, the communication hole may be used as the first communication hole or the second communication hole.

In a case where the first communication hole is provided in the stent, the loop-shaped connection member may be configured to pass through the outside of the pusher catheter and enter the pusher catheter through the second communication hole. In this case, while the connection member does not have the first intermediate portion and the second intermediate portion, the second end portion is located in the pusher catheter. Therefore, an effect that the operation force required for releasing the stent is less likely to increase is obtained.

The stent in the present invention is not limited to the aspect in which the above-described structure is provided. That is, the stent in the present invention may be a so-called pigtail type stent in which at least one end portion is rounded in a loop shape by coming-off of the tube 81, and it is not essential to include the flap 50.

What is claimed is:

1. A stent delivery apparatus comprising:
a guide catheter through which a guide wire is insertable;
a pusher catheter including a first lumen through which the guide catheter is inserted and a first communication hole communicating with the first lumen;
a stent including a second lumen through which the guide catheter is inserted and a second communication hole communicating with the second lumen and disposed on a distal side of the pusher catheter; and
a thread that connects the stent and the pusher catheter to each other in a releasable manner,
wherein the thread has
a first end portion that is supported by the pusher catheter through the first communication hole,
an intermediate portion that extends to a space between the stent and the pusher catheter through the second communication hole, and
a second end portion that extends around a part of the guide catheter located between a proximal end of the stent and a distal end of the pusher catheter.

2. The stent delivery apparatus according to claim 1,
wherein the intermediate portion has
a first intermediate portion that enters the second lumen through a proximal end opening of the stent and extends to the second communication hole, and
a second intermediate portion that extends from the second communication hole to a space between the stent and the pusher catheter through an outside of the stent.

3. The stent delivery apparatus according to claim 1,
wherein the second end portion passes through a loop shape at a position between the proximal end of the stent and the distal end of the pusher catheter.

4. The stent delivery apparatus according to claim 1,
wherein a state where the stent and the pusher catheter are connected to each other by the thread is maintained by the guide catheter passing through a loop shape between the proximal end of the stent and the distal end of the pusher catheter.

5. A stent delivery apparatus comprising:
a guide catheter through which a guide wire is insertable;
a pusher catheter through which the guide catheter is inserted;
a stent through which the guide catheter is inserted, the stent being disposed on a distal side with respect to the pusher catheter; and
a thread that connects the stent and the pusher catheter to each other in a releasable manner,
wherein one of the stent and the pusher catheter has a communication hole, and
the thread has
a first end portion that is supported by the other of the stent and the pusher catheter,
an intermediate portion that extends to at least a space between the stent and the pusher catheter through the communication hole, and
a second end portion that extends around a part of the guide catheter located on a proximal side with respect to a proximal end of the stent.

6. The stent delivery apparatus according to claim 5,
wherein the communication hole is formed in the stent.

7. The stent delivery apparatus according to claim 5,
wherein the communication hole is formed in the pusher catheter.
